(19) United States
(12) Patent Application Publication
(10) Pub. No.: US 2019/0167482 A1
(43) Pub. Date: Jun. 6, 2019

(54) ABSORBENT SHEET

(71) Applicant: Oji Holdings Corporation, Tokyo (JP)

(72) Inventors: Asami KAWAGUCHI, Tokyo (JP); Yuko SAITO, Tokyo (JP); Ayako TAKAHASHI, Tokyo (JP)

(73) Assignee: Oji Holdings Corporation, Tokyo (JP)

(21) Appl. No.: 16/313,968

(22) PCT Filed: May 30, 2017
(86) PCT No.: PCT/JP2017/020102

§ 371 Date: Dec. 28, 2018

(30) Foreign Application Priority Data
Jun. 29, 2016 (JP) 2016-128898

(51) Int. Cl. A61F 13/00; A61F 13/02; A61L 15/40

(52) U.S. Cl. CPC A61F 13/00063; A61F 13/0206; A61L 15/40; A61L 15/46; A61F 2013/00719; A61F 13/0256; A61F 13/00991

(57) ABSTRACT

The purpose of the present invention is to provide an absorbent sheet that can sufficiently absorb exudates exuding from the area of a lesion and can exhibit a powerful deodorizing effect for illness-associated odors. The present invention relates to an absorbent sheet with deodorizing functions comprising cellulosic fibers, absorbent fibers, a thermal adhesive resin and a deodorizing agent, in a single layer thereof.

### TECHNICAL FIELD

The present invention relates to an absorbent sheet. Specifically, the present invention relates to an absorbent sheet with deodorizing functions that has an absorbing function and a deodorizing function.

### BACKGROUND ART

Heretofore, it is known that a lesion in the skin may often give off distinctive odors (hereinafter this may be referred to as illness-associated odors). In particular, it is known that patients with advanced cancer by invasion in the skin from breast cancer, skin cancer, head and neck cancer and the like may give off sulfurous distinctive, illness-associated odors. When having strong odors associated with illness, patients may often worry about the odors and may have a risk of isolation as refusing to meet with other persons. In addition, medical service workers and patients' families must carry out nursing care in an environment of strong illness-associated odors, and may often assume a heavy burden.

As a measure for relieving such illness-associated odors, a lesion to give off illness-associated odors may be covered with a covering material. For example, Patent Document 1 discloses an illness odors-suppressing sheet for medical use having a nonwoven fabric infiltrated with a deodorant. In this, a deodorizing effect is expected by using a nonwoven fabric infiltrated with a deodorant. Patent Document 2 discloses a wound covering material having an absorbent layer. The wound covering material disclosed in Patent Document 2 is mainly to protect a wound region and to absorb a wound exudate, and also to prevent odor generation from a wound region. In Patent Document 2, the wound covering material has an activated carbon fabric layer in addition to the absorbent layer therein, thereby proposing absorption of the exudates and the odors from a wound region.

### CITATION LIST

### Patent Literature

Patent Document 1: JP-A-2014-113289
Patent Document 2: JP-A-2014-523778

### SUMMARY OF INVENTION

### Technical Problem

However, the illness odors-suppressing sheet for medical use described in Patent Document 1 would be effective in some degree for suppression of odors, but has a problem in that the sheet could not sufficiently absorb the exudates from the area of a lesion. The exudates from a lesion contain odors, and therefore if such exudates leak out from the sheet, they may give off odors to be a problem. In addition, when the exudates from the area of a lesion spread to the peripheral skin and tissue, there occurs another problem in that the area of the lesion is thereby enlarged.

The wound covering material described in Patent Document 2 has an absorbent layer and therefore can absorb exudates and odors from the area of a lesion, but as a result of investigations made by the present inventors, it has been clarified that the absorbing effect is not sufficient. In particular, sulfurous illness-associated malodors that may be given off by advanced cancer patients would be strong even though a few, and it is desired to improve the sheet so as to attain a sufficient deodorizing effect.

Given the situation, for the purpose of solving the technical problems in the related art, the present inventors have promoted investigations for providing an absorbent sheet that can sufficiently absorb fluids such as exudates and the like to leak from a lesion and can exhibit a powerful deodorizing effect for illness-associated odors.

### Solution to Problem

As a result of assiduous studies made for the purpose of solving the above-mentioned problems, the present inventors have found that an absorbent sheet can sufficiently absorb exudates exuding from the area of a lesion and can exhibit a powerful deodorizing effect for illness-associated odors by allowing the absorbent sheet to comprise cellulosic fibers, absorbent fibers, a thermal adhesive resin and a deodorizing agent. Specifically, the present invention has the following constitution.
[1] An absorbent sheet with deodorizing functions, comprising cellulosic fibers, absorbent fibers, a thermal adhesive resin and a deodorizing agent, in a single layer thereof.
[2] The absorbent sheet with deodorizing functions according to [1], wherein the content of the absorbent fibers is 3% to 50% by mass relative to the total mass of the solid content constituting the absorbent sheet with deodorizing functions.
[3] The absorbent sheet with deodorizing functions according to [1] or [2], wherein the absorbent fibers contain a sodium polyacrylate resin.
[4] The absorbent sheet with deodorizing functions according to any one of [1] to [3], wherein the deodorizing agent contains a component capable of reacting with at least one selected from sulfides, sulfurs and amines.
[5] The absorbent sheet with deodorizing functions according to any one of [1] to [4], wherein the deodorizing agent contains at least one selected from metals, minerals, activated carbons and vegetable extracts.
[6] The absorbent sheet with deodorizing functions according to any one of [1] to [4], wherein the deodorizing agent is a betaine compound.
[7] The absorbent sheet with deodorizing functions according to any one of [1] to [6], further having a resin layer containing a deodorizing agent.
[8] The absorbent sheet with deodorizing functions according to any one of [1] to [7], further having a surface protective layer.
[9] The absorbent sheet with deodorizing functions according to any one of [1] to [8], further having a surface contact layer.
[10] The absorbent sheet with deodorizing functions according to [9], wherein the surface contact layer is a non-adhering layer.
[11] The absorbent sheet with deodorizing functions according to any one of [1] to [10], which is for covering an area to give off illness-associated odors.

### ADVANTAGEOUS EFFECTS OF INVENTION

According to the present invention, there can be obtained an absorbent sheet with deodorizing functions that can sufficiently absorb exudates exuding from the area of a lesion and can exhibit a powerful deodorizing effect for illness-associated odors. In addition, the absorbent sheet with deodorizing functions of the present invention can prevent the absorbed exudates from spreading in the planar direction of the sheet and therefore can prevent the exudates and the like from being brought into contact with the peripheral area around a lesion, and further can prevent the once-absorbed fluids from turning back.

### BRIEF DESCRIPTION OF DRAWINGS

FIG. 1 is a schematic cross-sectional view for explaining a configuration of one example of an absorbent sheet with deodorizing functions of the present invention.

FIG. 2 is a schematic cross-sectional view for explaining a configuration of one example of an absorbent sheet with deodorizing functions of the present invention.

FIG. 3 is a schematic cross-sectional view for explaining a configuration of one example of an absorbent sheet with deodorizing functions of the present invention.

FIG. 4 is a schematic cross-sectional view for explaining a configuration of one example of an absorbent sheet with deodorizing functions of the present invention.

FIG. 5 is a schematic view for explaining a configuration of an apparatus for forming an absorbent sheet with deodorizing functions of the present invention.

### DESCRIPTION OF EMBODIMENTS

The present invention is described in detail hereinunder. The description of the constitutive elements of the invention given hereinunder is for some typical embodiments and examples of the invention, but the invention should not be limited to such embodiments. In this description, the numerical range expressed by the wording “a number to another number” means the range that falls between the former number indicating the lower limit value of the range and the latter number indicating the upper limit value thereof.

(Absorbent Sheet with Deodorizing Functions)

The present invention relates to an absorbent sheet with deodorizing functions comprising cellulosic fibers, absorbent fibers, a thermal adhesive resin and a deodorizing agent, in a single layer thereof.

In the absorbent sheet with deodorizing functions of the present invention, the cellulosic fibers and the absorbent fibers are bonded and fixed via at least a part of the thermal adhesive resin. The thermal adhesive resin has a function of melting by heat to bond and fix the components of the sheet. In addition, the thermal adhesive resin may be thermal adhesive resin fibers, and may also be fibrillated fibers.

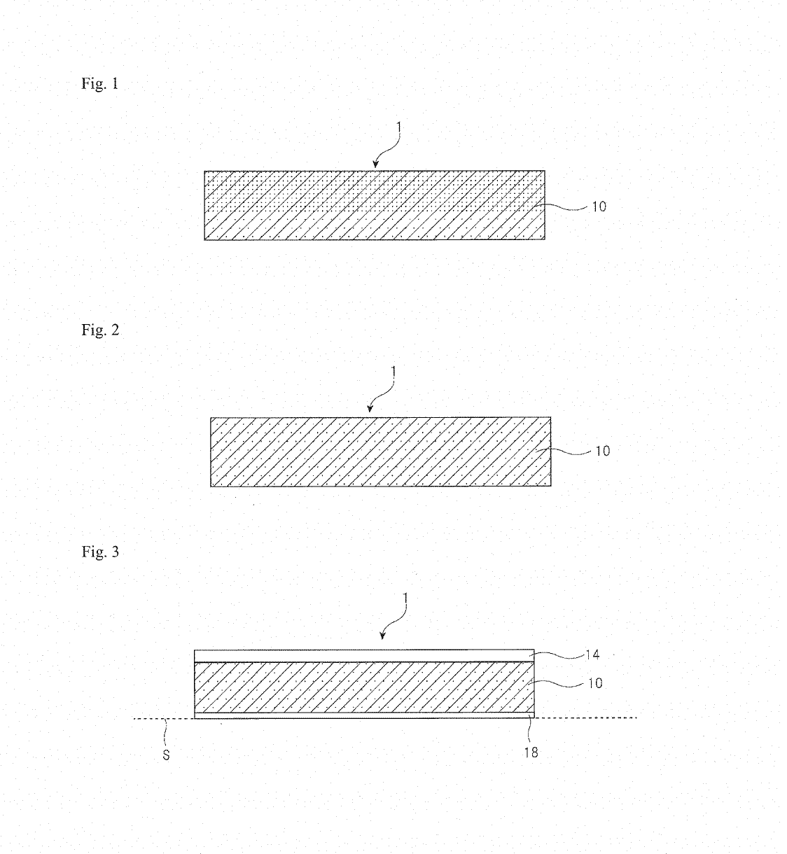

FIG. 1 is a schematic cross-sectional view for explaining a configuration of one example of an absorbent sheet with deodorizing functions of the present invention. As shown in FIG. 1, the absorbent sheet with deodorizing functions 1 of the present invention is preferably a single layer structure consisting of a sheet substrate 10. Such an absorbent sheet with deodorizing functions 1 (sheet substrate 10) contains cellulosic fibers, absorbent fibers, a thermal adhesive resin and a deodorizing agent therein. Besides, the absorbent sheet with deodorizing functions 1 of the present invention may be a laminate formed by laminating two or more layers containing cellulosic fibers, absorbent fibers, a thermal adhesive resin and a deodorizing agent.

In the absorbent sheet with deodorizing functions of the present invention, fiber components such as cellulosic fibers, absorbent fibers, a thermal adhesive resin and a deodorizing agent may be entirely dispersed. Among others, preferably, cellulosic fibers, absorbent fibers and a thermal adhesive resin are entirely dispersed, and more preferably, cellulosic fibers and absorbent fibers are entirely dispersed. Herein, entire dispersion of fiber components means that when the absorbent sheet with deodorizing functions is divided into 5 regions in the thickness direction, a difference in the absolute values of the average concentrations of components in adjacent regions is 10% by mass or less.

In the absorbent sheet with deodorizing functions of the present invention, the deodorizing agent may he unevenly distributed to either one surface side. In FIG. 1 and FIG. 2, for clear understanding of the state of dispersion of the deodorizing agent, the deodorizing agent is conceptually indicated with black points. In. FIG. 1, the deodorizing agent is unevenly distributed to one surface side of the sheet, and this aspect is also preferable. In FIG. 2, the deodorizing agent is uniformly dispersed in the absorbent sheet with deodorizing functions. In the absorbent sheet with deodorizing functions 1, cellulosic fibers, absorbent fibers, a thermal adhesive resin, and a deodorizing agent may be uniformly dispersed. In the aspect as shown in FIG. 1, when the absorbent sheet with deodorizing functions is divided into 5 regions in the thickness direction, a difference in the concentrations of the deodorizing agents in adjacent regions is not particularly limited, and it may be 10% by mass or more, or may also be 20% by mass or more.

In the absorbent sheet with deodorizing functions of the present invention, the thermal adhesive resin may also be unevenly distributed to either one surface side, as with the deodorizing agent. As described later, there is a case where the thermal adhesive resin powders and the deodorizing agent (particle mixture) are spayed after accumulation of the fiber mixture. In such a case, the particle mixture may be unevenly distributed to one surface side. When the absorbent sheet with deodorizing functions is divided into 5 regions in the thickness direction, a difference in the concentrations of the particle mixtures in adjacent regions is not particularly limited, and it may be 10% by mass or more, or may also be 20% by mass or more.

Absorbent fibers are used in the absorbent sheet with deodorizing functions of the present invention, and the absorbent fibers are uniformly dispersed in the absorbent sheet with deodorizing functions. Thus, the present absorbent sheet can exhibit an excellent fluid (exudate or the like) absorbing performance and a deodorizing effect. In addition, in such an aspect, the absorbent sheet enables spot absorption, therefore preventing the absorbed fluids from spreading or turning back.

Moreover, in the absorbent sheet with deodorizing functions of the present invention, since a component capable of exhibiting an absorbing function is present close to a component capable of exhibiting a deodorizing function, for example, odorous components contained in the absorbed fluid can be promptly deodorized. Furthermore, since the absorbent sheet with deodorizing functions of the present invention has the above-described configuration, it is also advantageous in that the production efficiency of the sheet itself is high.

The absorbent fibers are composed of, for example, an absorbent polymer. The absorbent fibers are formed by processing such an absorbent polymer to fibers, and the advantages thereof are that the absorbent polymer constituting the absorbent fibers can be prevented from being unevenly located in the absorbent sheet with deodorizing functions, and further that, by using the absorbent fibers, the absorbent polymer constituting the absorbent fibers can be prevented from dropping off or flowing away from the absorbent sheet with deodorizing functions, thereby increasing the absorption performance in the absorbent sheet with deodorizing functions.

In the absorbent sheet with deodorizing functions of the present invention, the cellulosic fibers are entangled with the absorbent fibers to form a net-like structure. Further, in a case where the thermal adhesive resin is also fibrous, the cellulosic fibers, the absorbent fibers and the thermal adhesive resin fibers are entangled to form a net-like structure. In addition, the absorbent sheet with deodorizing functions having a net-like structure can exhibit powerful absorption performance not only for fluids having a low viscosity but also for fluids having a high viscosity.

Moreover, in the present invention, the absorbent sheet with deodorizing functions having such a net-like structure can exhibit a more excellent deodorizing function. This may be considered to be because the absorbent fibers absorb malodorous substances and, in addition, the net-like structure itself can trap malodorous substances to exhibit a deodorizing function.

As described above, the absorbent sheet with deodorizing functions of the present invention can exhibit an excellent fluid absorbing performance and a deodorizing effect. In addition, the sheet can prevent absorbed fluids from spreading and turning back. Consequently, the absorbent sheet with deodorizing functions of the present invention is favorable for covering an area that may give off illness-associated odors. Specifically the sheet can be used for covering the area of a lesion caused by invasion in the skin from breast cancer, skin cancer, head and neck cancer, etc. In addition, apart from the above-mentioned use, the sheet can also be used for covering the area of bedsores, or as a constituent member for diapers. Further, the sheet can also be used for bedclothes such as bed sheets, etc., and for bag-like storage cases, etc.

The absorbent sheet with deodorizing functions of the present invention is preferably formed according to an air-laid method. Here, the air-laid method is a type of a method of forming a nonwoven fabric in dry. Specifically, according to this method, fibers constituting the absorbent sheet with deodorizing functions are defibrillated in dry and are then deposited on a wire fabric running along with air serving as a medium, while being mixed with the defibrillated fibers and various components, so as to form a web (nonwoven sheet) thereon. The fibers constituting the web are bonded to each other according to various bonding methods. In that manner, the absorbent sheet with deodorizing functions of the present invention is preferably a nonwoven fabric formed according to the air-laid method (air-laid nonwoven sheet)

The air-laid nonwoven fabric sheet can be controlled, so that the sheet becomes bulky and the space between material fibers becomes large. By increasing the space between material fibers in the air-laid nonwoven fabric sheet, absorption performance or deodorizing performance can be easily exhibited, so that a sheet having excellent absorption performance can be obtained.

The extent of absorption in the absorbent sheet with deodorizing functions is preferably 1500 g/m² or more, more preferably 2500 g/m², even more preferably 4000 g/m² or more, and particularly preferably 5000 g/m² or more. The extent of absorption means an amount of water remaining in the absorbent sheet with deodorizing functions, after the sheet is immersed in a physiological saline solution for 10 minutes and then let the sheet drip for 1 minute.

The density, the basis weight, the vapor permeability and the like of the absorbent sheet with deodorizing functions of the present invention are not particularly limited. The density, the basis weight, the vapor permeability and the like of the absorbent sheet with deodorizing functions of the present invention may he controlled depending on the use and the function thereof.

### <Cellulosic Fibers>

As the cellulosic fibers, various cellulosic fibers conventionally used in absorbents can be employed. Examples of the material of the cellulosic fibers include pulp, rayon, cotton, and cupra. Above all, pulp fibers are preferably used as the cellulosic fibers, and pulp fibers are excellent in point of fiber length, productivity, raw material cost, etc. One type alone or two or more types of cellulose fibers may be used either singly or as combined.

Examples of the pulp fibers include those obtained from raw material pulp such as wood pulp (softwood, hardwood), or non-wood pulp such as rag pulp, linter pulp, linen pulp, kozo (mulberry tree) pulp, mitsumata (paper bush) pulp, ganpi (bark of clove-like bush) pulp, etc.; recycled waste-paper pulp, etc. Depending on the manufacturing method thereof, pulp may be grouped into categories of mechanical pulp (GP, RGP, TMP) and chemical pulp (sulfate pulp, kraft pulp). Above all, wood pulp is preferably used for pulp fibers, as excellent in supply, quality stability, cost, etc.

The mass-average fiber length of the pulp fibers is not particularly limited, but it is preferably 0.05 to 5 mm. By setting the fiber length the pulp fibers within the above-described range, the pulp fibers can be prevented from dropping off from the absorbent sheet with deodorizing functions and can enhance the fluid absorption efficiency of the absorbent sheet.

The content of the cellulosic fibers in the absorbent sheet with deodorizing functions is not particularly limited, but it is preferably 1% to 80% by mass relative to the total mass of the solid content constituting the absorbent sheet with deodorizing functions. By setting the content of the cellulosic fibers within the above-described range, the fluid absorption performance of the absorbent sheet can be further enhanced.

### <Absorbent Fibers>

The absorbent fibers (SAF) are obtained by fibrillation of an absorbent polymer (SAP) that absorbs water and swells. Herein, the absorbent polymer (SAP) is preferably a superabsorbent polymer, and “superabsorbent” means that the polymer can absorb water in an amount of 20 times or more the self-weight. The absorbent polymer (SAP) includes starch-type, cellulosic, synthetic resin-type polymers, etc., such as starch-acrylic acid (salt) graft copolymer, isobutylene-maleic acid copolymer, saponified starch-ethyl acrylate graft copolymer, saponified starch-methyl methacrylate graft copolymer, saponified starch-acrylonitrile copolymer, saponified starch-acrylamide graft copolymer, acrylic acid (salt) polymer, acrylic acid-crosslinked polyethylene oxide, crosslinked sodium carboxymethyl cellulose, crosslinked polyvinyl alcohol-maleic anhydride copolymer, amino acid-crosslinked biodegradable polyaspartic acid, culture product from Alcaligenes latus, etc. Above all, acrylic acid (salt) polymer is preferably used, and in particular, sodium polyacrylate-type resin is preferably used. Specifically, the absorbent fibers used in the present invention preferably contain a sodium polyacrylate resin. The absorbent fibers may be fibrillated fibers or nanofibers, or may also be a mixture thereof

The content of the absorbent fibers contained in the absorbent sheet with deodorizing functions is not particularly, but it is preferably 1% to 70% by mass, and more preferably 3% to 50% by mass, relative to the total mass of the solid content constituting the absorbent sheet with deodorizing functions, By setting the content of the absorbent fibers within the above-described range, the fluid absorbability of the absorbent sheet with deodorizing functions can be effectively increased. In addition, by setting the content of the absorbent fibers within the above-described range, the absorbent sheet can prevent any unintentional moisture absorption in use environments.

### Thermal Adhesive Resin

The thermal adhesive resin is a binder component for binding the cellulosic fibers with the absorbent fibers. At least a part of the thermal adhesive resin is melted by heating, and the molten resin is then solidified to bind the cellulosic fibers with the absorbent fibers. Examples of such a thermal adhesive resin include synthetic fibers of polyethylene (including copolyethylene), polypropylene (including modified polypropylene, and copolypropylene), low-melting-point polyester (for example, low-melting-point polyethylene terephthalate), low-melting-point polyamide, low-melting-point polylactic acid, polybutylene succinate, acrylic resin, urethane resin, styrene-butadiene copolymer, polyvinyl alcohol (PVA), and ethylene-vinyl acetate copolymer (EVA).

The thermal adhesive resin may be in the form of fibers consisting of a single resin, or may also be a composite resin of a combination of two or more types of resins. For example, a core/sheath type composite resin is preferably used, which is formed of a core part consisting of a resin with a high inciting point, and a sheath part consisting of a resin with a low inciting point that covers the outer periphery of the core part. The composite form of a combination of two types of resins differing in the melting point include PET/PET composite resin, PE/PE composite resin, PP/PP composite resin, EVA/PP composite resin, PE/PET composite resin, PP/PET composite resin, and PE/PP composite resin. Above all, a core; sheath type composite resin formed of a core part of polypropylene fibers (melting point 160° C.) and a cover layer of polyethylene (melting point 130° C.) (PE/PP composite resin) and a core/sheath type composite resin formed of a core part of PET fibers and a cover layer of polyethylene (PE/PET composite resin) are preferably used. In addition, those having crimped fibers are also preferable as readily, giving a soft sheet. In a case where such a core/sheath type composite resin is used, preferably, the cover layer alone is melted by applying hot air thereto at a temperature at which the outer cover layer melts but the core part does not melt (as one example, at 140° C.). In this case, the core part is not melted and therefore can remain as stable fibers. Accordingly, the voids are not damaged in melting and bonding, and therefore a net-like structure can be easily formed.

In addition, in the present invention, a side-by-side type composite resin may also he used, in which two types of resins differing in the melting point are arranged to be adjacent to each other.

The form of the thermal adhesive resin is not particularly limited, and it may be any of a powdery, granular or fibrous form, or may also be a combination thereof combined in any desired manner. Above all, the thermal adhesive resin is preferably fibrous. Such thermal adhesive resin fibers are favorably used since they have both functions of a thermal adhesive resin and fibers by themselves. The thermal adhesive resin fibers may be crimped, curled or spiraled, or may be fibrillated, like pulp fibers.

In a case where the thermal adhesive resin is powdery or granular, the mean particle size thereof is not particularly limited. Also, in a case where the thermal adhesive resin is fibrous, the mass-average fiber length and fiber diameter of the thermal adhesive resin fibers are not particularly limited, either, and it may be adequately selected within a range capable of exhibiting the advantageous effects of the present invention.

The content of the thermal adhesive resin is not particularly limited, but it is preferably 1% to 70% by mass relative to the total mass of the solid content constituting the absorbent sheet with deodorizing functions.

### <Deodorizing Agent>

The deodorizing agent is preferably an agent capable of exhibiting a deodorizing effect to various odors. That is to say, the absorbent sheet with deodorizing functions of the present invention is preferably an absorbent sheet capable of exhibiting a deodorizing effect to a variety of odor-emitting substances. Among others, the deodorizing agent preferably contains a component capable of reacting with a causative compound that may give off illness-associated odors. Herein., examples of the causative compound that may give off illness-associated odors include nitrogen-containing compounds, sulfur-containing compounds, amine compounds, phenols, and lower fatty acids. Specifically, the illness-associated odor-emitting causative compound includes, but are not limited to, trimethylamine, isovaleric acid, dimethyl trisulfide, methyl mercaptan, cadaverine, putrescine, diacetyl, and lower fatty acid. Above all, the deodorizing agent used in the present invention preferably contains a component capable of reacting with at least one selected from nitrogen-containing compounds and sulfur-containing compounds, and more preferably contains a component capable of reacting with at least one selected from sulfides, sulfurs and amities Further, the deodorizing agent used in the present invention preferably contains a component capable of reacting with sulfides, and above all, preferably contains a component capable of reacting with at least one selected from dimethyl trisulfide, methyl mercaptan, and diamines such as cadaverine or putrescine.

Herein, the phrase “reacting with” the above-mentioned compound means that (a) the deodorizing agent chemically reacts with the illness-associated odor-emitting causative compound to thereby change the illness-associated odor-emitting causative compound into any other having a structure not emitting odors, (b) the deodorizing agent physically absorbs the illness-associated odor-emitting causative compound, or (c) the deodorizing agent masks the odors of the illness-associated odor-emitting causative compound. According to such a “reaction”, the deodorizing agent can exhibit the deodorizing effect. For example, the deodorizing agent that chemically reacts with an illness-associated odor-emitting causative compound includes a deodorizing agent containing a vegetable extract, polyphenol and polyphenol oxidase; and the deodorizing agent that physically absorbed an illness-associated odor-emitting causative compound includes metals and minerals, for example, zeolite, silver ion-containing zeolite, etc., as well as activated carbon. The deodorizing agent capable of masking the odors of an illness-associated odor-emitting causative compound includes a deodorizing agent containing an aromatic component. Above all, for illness-associated odor-emitting causative compounds of sulfides, the use of a deodorizing agent containing any of metals, minerals, activated carbons, vegetable extracts, polyphenols and polyphenol oxidases is more preferable.

The deodorizing agent may also be an agent that decomposes microbes such as bacteria, fungi, yeasts and others, or prevents the growth of such microbes to thereby suppress malodors. Such a deodorizing agent includes zinc pyrithione, zinc oxide, chitin, chitosan, organic nitrogenous/sulfurous compounds, escallops, and substances having a microbicidal (antimicrobial)/fungicidal. (antifungal) function.

In the present invention, the use of a deodorizing agent containing at least one selected from metals, minerals, activated carbons and vegetable extracts is preferable. In addition, the use of a deodorizing agent containing polyphenol and polyphenol oxidase is also preferable. The polyphenol that can be contained in the deodorizing agent is a compound having two or more hydroxyl groups substituted for the hydrogen atoms on the same single benzene ring, and a glycoside thereof is also included in the polyphenol. In addition, the polyphenol may be a polymer.

The polyphenol that can be contained in the deodorizing agent is not particularly limited, but above all, polyphenol having a hydroquinone or o-diphenol structure is preferable. The o-diphenol structure means a structure in which a benzene ring is directly substituted with hydroxyl groups and the hydroxyl groups are adjacent to each other.

Specific examples of polyphenols include apigenin, apigenin glycoside, acacetin, isorhamnetin, isorhamnetin glycoside, isoquercitrin, epicatechin, epicatechin gallate, epigallocatechin, epigallocatechin gallate, aesculetin, ethylprotocatechuate, ellagic acid, catechol, gamma acid, catechin, gardenine, gallocatechin, caffeic acid, caffeate, chlorogenic acid, kaempferol, kaempferol glycoside, quercetin, quercetin glycoside, quercetagenin, genisetin, genisetin glycoside, gossypetin, gossypetin glycoside, gossypol, 4-dihydroxyanthraquinone, 1,4-dihydroxynaphthalane, cyanidine, cyanidine glycoside, sinensetin, diosmetin, diosinetin glycoside, 3,4′-diphenyldiol., sinapinic acid, stearyl-β-(3,5-di-t-butyl-4-hydroxyphenyl)propionate, spinaeetin, tangeretin, taxifolin, tannic acid, daphnetin, tyrosine, delphinidin, delphinidin glycoside, theaflavin, theaflavin monogallate, theaflavin bisgallate, tricetinidin, dopa, dopamine, naringenin, naringin, nordihydrogual arctic acid, noradrenalin, hydroquinone, vanillin, patchouletin, herbacetin, vanillyl alcohol, vanitrope, vanillin propylene glycol acetal, vanillic acid, bis(4-hydroxyphenyl)sulfonic acid, bisphenol A, pyrocatechol, vitexin, 4,4′-biphenyldiol, 4-t-butylcatechol, 2-tert-butylhydroquinone, protocatechuic acid, phloroglucinol, phenolic resin, procyanidin, prodelphinidin, phloretin, phloretin glycoside, fisetin, folin, ferbacetin, fraxetin, phloridin, peonidin, peonidin glycoside, pelargonidin, pelargonidin glycoside, petunidin, petunidin glycoside, hesperetin, hesperidin, gallic acid, gallate (lauryl gallate, propyl gallate, butyl gallate), mangeferin, malvidin, malvidin glycoside, myricetin, myricetin glycoside, 2,2′-mehtylenebis(4-methyl-6-t-butylphenol), 2,2′-methylenebis(4-ethyl-6-t-butylphenol), 2,2′-methylenebis(4-methyl-6-tert-butylphenol), methylatrarate, 4-methylcatechol, 5-methylcatechol, 4-niethoxycatechol, 5-methoxycatechol, methylcatechol-4-carboxylic acid, 2-methylresorcinol, 5-methylresorcinol, morin, limocitrin, limocitrin glycoside, limocitrol, luteolin, luteolin glycoside, luteolinidin, luteolinidin glycoside, rutin, resorcin, resveratrol, resorcinol, leucocyanidin, and leucodelphinidin. These polyphenols may be used either singly or in combination of two or more types,

The above-described polyphenols may be prepared according to known methods, but commercially available products may also be purchased. In addition, they may be prepared through synthesis. Further, high-concentration polyphenol fractions prepared from vegetables can also be used.

Preferred examples of polyphenol oxidase include oxidases contained in vegetable extracts. Specific examples of such polyphenol oxidase include oxidases contained in burdock extract, coffee bean extract, and green tea leaf extract. Oxidase contained in burdock extract is a preferred example.

As the deodorizing agent, commercially available products can be used. Specifically, DEOATAK (registered trademark) manufactured by Takasago International Corporation and others can be used.

Examples of metals include Cu, Fe, Ni, Zn, Ag, Ti, Co, Al, Cr, Pb, Sri, In, Zr, Mo, Mn, Cd, Bi, and Mg. Preferably, the metal exists as metal fine particles and/or metal ions.

Minerals are hydrous silicates containing a metal such as aluminum, iron, manganese, magnesium, calcium or potassium, sodium, and are aggregates of fine crystal pieces. Examples of the minerals include smectites (montmorillonite, beidellite, nontronite, saponite, hectorite, sauconite, stevensite, etc.), micas (sericite, illite, muscovite, phlogopite, etc.), vermiculite, chlorite, pyrophyllite, talc, kaolin minerals, sementinite, sepiolite, allophane, zeolite, and hydrotalcite. In addition, metal oxides such as silicon dioxide or calcium oxide can also be used.

Examples of activated carbon include coconut carbon, coal, and wood coal.

Examples of vegetable extracts include extracts extracted with a solvent or the like from turmeric, gambir, common sage, tea, rosemary, fennel, thyme, nutmeg, pepper, turmeric, vanilla, bell pepper, coix seed, bupleurum root, Japanese quince, sapanwood, culen, John love, oxalis, chameleon plant, hemlock, ginkgo, Japanese black pine, larch, Japanese red pine, empress tree, fortune tea olive, lilac, orange osmanthus, sweet coltsfoot, crested leopard, forsythia, chestnut, alder, quercus serrata, pomegranate, fig, flowering fern, weigela, persimmon, fleawort, mugwort, mountain maple, crape myrtle, Lespedeza thunhergii var. albiflora, Japanese andromeda, fern, aspen, Japanese ash, sweet potato (sucrose), eucalyptus, etc.

In addition, examples of organic deodorizing agents other than vegetable extracts include deodorizing agents derived from organic acids such as amino acids, or from natural products, vegetables or food materials. For example, cyclodextrin and the like can also be used. Regarding such cyclodextrin, α- β- or γ-type cyclodextrin may be selected or mixed depending on the intended use.

In the present invention, a betaine compound can also be used as a deodorizing agent. The betaine compound includes a betaine compound and a derivative of the betaine compound. The betaine compound chemically reacts with an illness-associated odor-emitting causative compound, so that it can convert the illness-associated odor-emitting causative compound to a structure that does not emit odors, thereby exhibiting a deodorizing effect.

The betaine compound is preferably a quaternary ammonium carboxylate-type compound. Among others, the betaine compound is preferably a compound represented by the following general formula (A):

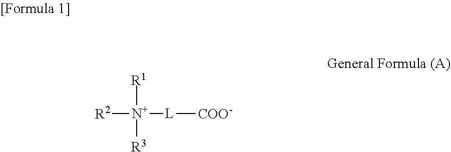

In the general formula (A), R¹ to R³ each independently represent a hydrogen atom or an alkyl group optionally having a substituent. The number of carbon atoms possessed by the alkyl group is preferably 1 to 20, and more preferably 1 to 10, In addition, in the general formula (A), L represents a single bond or a divalent linking group. The divalent linking group is preferably an alkylene group optionally having a substituent, and the number of carbon atoms possessed by the alkylene group is preferably 1 to 10, and more preferably 1 to 5.

As such a betaine compound, for example, a commercially available product, “Epoleon” manufactured by Kyoritsuseiyaku Corporation can be used. Specifically, Epoleon X-100, Epoleon X-110, Epoleon POW-22, and the like are preferably used.

As a deodorizing agent, the aforementioned deodorizing agent may he used alone, but a plurality of deodorizing agents may also be used in combination. Moreover, deodorizing agents other than the above-described deodorizing agents may also be used in combination. By using multiple types of deodorizing components in combination, various illness-associated odor-emitting causes can be addressed.

The shape of the deodorizing agent may be a powder, a granule, a liquid or a fiber, and thus, it is not particularly limited.

### <Other Components>

The absorbent sheet with deodorizing functions may be composed of the above-mentioned constituent components alone, but may contain any other components as needed. Examples of such other components include functional powder, functional fibers, and functional liquid. The functional powder is preferably one having any one or more functions of antiallergenic function, antiviral function, aromatic function, etc. Examples of the functional powder include titanium oxide, titanium dioxide, magnesium oxide, vegetable extract, flavonol, collagen fibers, iron oxide, citric acid, tannic acid, hinokithiol, and eucalyptus extract. The functional fibers and the functional liquid may be fibers or liquid containing the above-mentioned functional powder.

(Layer Configuration of Absorbent Sheet with Deodorizing Functions)

The absorbent sheet with deodorizing functions of the present invention preferably further has a surface protective layer. As shown in FIG. 3, the absorbent sheet with deodorizing functions 1 of tile present invention may further have a surface protective layer 14. In this ease, the absorbent sheet with deodorizing functions 1 has the surface protective layer 14 on one surface of a sheet substrate 10. The surface protective layer 14 may be a nonwoven fabric, but is preferably a waterproof film. The surface protective layer 14 that is a waterproof film can protect the surface that is an exposed surface during use from wetting with water, etc., and therefore can more effectively exhibit liquid absorbability and a deodorizing effect. In addition, exchange frequency of the absorbent sheet with deodorizing functions can be reduced. Further, the waterproof film is preferably pervious to vapor (pervious to moisture) as capable of preventing a lesion from getting stuffy.

The surface protective layer 14 includes a waterproof film, a moisture-pervious film, and an unwoven fabric (for example, a water-repellent unwoven fabric). The waterproof film is, for example, a PE film. The nonwoven fabric includes a spunbond nonwoven fabric, a spunlace nonwoven fabric, a thermal bonded nonwoven fabric, and an SMS (spunbond/melt-blown/spunbond) nonwoven fabric.

Preferably, the absorbent sheet with deodorizing functions of the present invention further has a surface contact layer. As shown in FIG. 3, the absorbent sheet with deodorizing functions 1 of the present invention preferably has a surface contact layer 18 on the side to be kept in contact with a skin surface S. The surface contact layer 18 is a layer to be in contact with an illness-associated odor emission area such as a wounded area, or a lesion area. Accordingly, the surface contact layer 18 is preferably formed of a material that does not irritate a wounded area and others and that does not stick to a wounded area.

The surface contact layer 18 has a structure capable of transferring fluids and odor-emitting causative substances, such as exudates and the like exuding from a wounded area, to the absorbent sheet with deodorizing functions 1. Specifically, the surface contact layer 18 includes a moisture-pervious film, a nonwoven fabric (for example, a water-repellent nonwoven fabric), a resin film, a resin net, etc. The nonwoven fabric includes a spunbond nonwoven fabric, a spunlace nonwoven fabric, a thermal bonded nonwoven fabric, and an SMS (spunbond/melt-blown/spunbond) nonwoven fabric. The resin film is, for example, a PET film. As the resin net, for example, Delnet X550 WHITE-E (manufactured by DelStar Incorporation) and Polynet (manufactured by Smith & Nephew KK) can be used. In the case of using a resin film, preferably, the resin film is perforated for securing fluid permeability.

The surface contact layer 18 is preferably a non-adhesive layer. When the surface contact layer 18 is a non-adhesive layer, it can prevent the absorbent sheet with deodorizing functions from adhering (sticking) to a wounded area. When the surface contact layer 18 is a non-adhering layer, it reduces irritation to be given to the wounded area when the absorbent sheet with deodorizing functions is peeled from a wounded area.

Preferably, the non-adhering layer is formed, for example, by providing fine pores through the surface contact layer 18. Use of a net-like sheet, a perforated film or an embossed sheet as a non-adhering layer is also a preferred embodiment. In such a manner, the structure of the non-adhering layer is preferably so planned that the contact area thereof to a skin surface S is reduced. In addition, fluid permeability is also needed, so that the absorbent sheet can absorb exudates exuding from a wounded area. Besides, since such exudates exuding from a wounded area include purulent exudates, fluid permeability for such purulent exudates is also needed. Beyond that, as the method of forming the non-adhering layer, a method of incorporating an oily component such as Vaseline into the layer, a method of vapor-depositing a component not adhering to skin such as aluminum or the like on the layer, or a method of incorporating or applying silicone to the layer is also preferable.

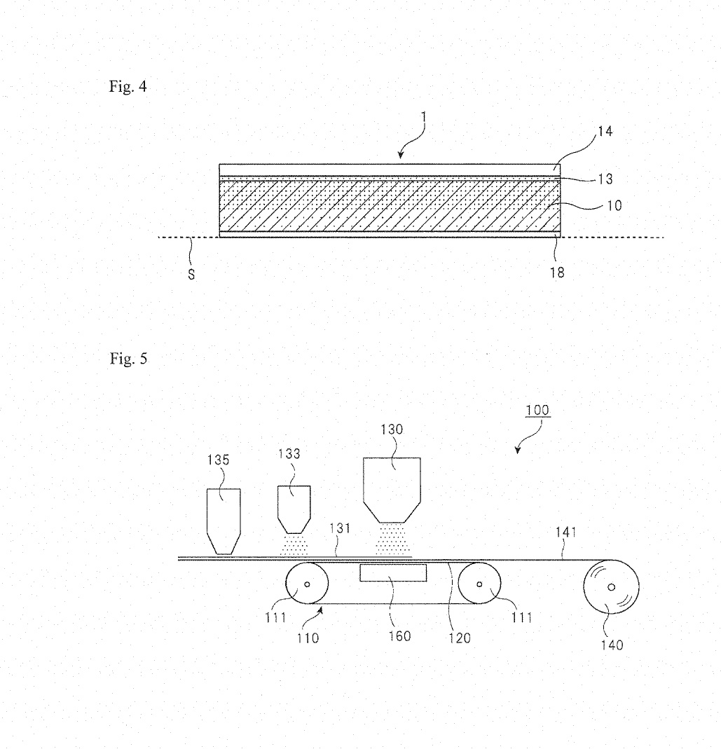

The absorbent sheet with deodorizing functions of the present invention may further have a resin layer containing a deodorizing agent. As shown in FIG. 4, the absorbent sheet with deodorizing functions 1 of the present invention may have a resin layer 13 containing a deodorizing agent on one surface of the sheet substrate 10. In this case, at least a portion of the deodorizing agent is incorporated into fibers constituting the sheet substrate 10, but the remaining portion of the deodorizing agent is contained in a resin layer laminated on the sheet substrate 10. In a case where the absorbent sheet with deodorizing functions 1 of the present invention further has a surface protective layer 14, the surface protective layer 14 is laminated on the resin layer 13.

The resin layer 13 comprises a thermal adhesive resin and a deodorizing agent. An example of the thermal adhesive resin may be a thermal adhesive resin contained in the sheet substrate 10. Examples of the thermal adhesive resin include polyethylene, polypropylene, polyethylene terephthalate (PET) such as low-melting-point polyethylene terephthalate, an ethylene-vinyl acetate copolymer (EVA), low-melting-point polyamide, low-melting-point polylactic acid, and polybutylene succinate. The thermal adhesive resin used to form the resin layer 13 is preferably a resin powder, and a resin powder melted by heating in the production step is preferable. Among others, a polyethylene resin powder is preferably used, but is not limited thereto.

The absorbent sheet with deodorizing functions of the present invention may further have any other layer than the above-mentioned layers. Such other layer is, for example, a vapor-pervious carrier sheet. Examples of the vapor-pervious carrier sheet include, but are not particularly limited to, a tissue paper layer, spunbond fabrics, and spunlace fabrics. However, the examples are not particularly limited thereto, as long as they are pervious to vapor. The vapor-pervious carrier sheet is, for example, a supporting layer in forming the absorbent sheet with deodorizing functions, and it is essential in manufacture. After the sheet formation, the vapor-pervious carrier sheet may be left as is, or may be peeled off Within a range not impairing the advantageous effects of the present invention in that manner, any layer may be laminated.

In addition, in laminating the above-mentioned layers and regions, if desired, an adhesive layer may be provided between the layers.

The total thickness of the absorbent sheet with deodorizing functions of the present invention is not particularly limited, and it is preferably 1 cm or less, and more preferably 0.5 cm or less, when a load of 7 gf/cm² is applied thereto. If the total thickness is too large, the adhesiveness to the affected area is likely to deteriorate. On the other hand, if the total thickness is too small, the extent of absorption is likely to decrease. The total thickness of the absorbent sheet with deodorizing functions may be adequately adjusted depending on the intended use of the absorbent sheet with deodorizing functions.

(Method for Producing Absorbent Sheet with Deodorizing Functions)

The present invention also relates to a method for producing the above-mentioned absorbent sheet with deodorizing functions. One preferred embodiment of the method for producing the absorbent sheet with deodorizing functions of the present invention includes a step of mixing cellulosic fibers, absorbent fibers, a thermal adhesive resin and a deodorizing agent in air and depositing the resultant mixture onto a vapor-pervious carrier sheet to form a web (first embodiment). Moreover, another embodiment of the method for producing the absorbent sheet with deodorizing functions includes a step of mixing cellulosic fibers, absorbent fibers and a thermal adhesive resin in air and depositing the resultant mixture onto a vapor-pervious carrier sheet to form a web, and a step of adding a deodorizing agent to the web (second embodiment).

In the step of forming a web in the aforementioned first embodiment, cellulosic fibers, absorbent fibers, a thermal adhesive resin and a deodorizing agent are mixed with one another in air (mixing step). An example of the mixing step is a step of mixing cellulosic fibers, absorbent fibers, a thermal adhesive resin and a deodorizing agent with one another to obtain a raw material for web. In the step of forming a web, it is preferable that cellulosic fibers, absorbent fibers, a thermal adhesive resin and a deodorizing agent be mixed with one another in air, and that the resultant mixture be then deposited on a vapor-pervious carrier sheet to form a web, but the web-forming step is not limited thereto. Upon performing the mixing step, a stirrer may be used, but it is preferable to mix the components using an air flow. In addition, in the mixing step, individual fibers are preferably defibrillated. By defibrillating individual fibers, a high-bulk sheet can be formed.

In the step of forming a web in the aforementioned second embodiment, cellulosic fibers, absorbent fibers and a thermal adhesive resin are first mixed with one another in air (mixing step). The mixing step is a step of mixing cellulosic fibers, absorbent fibers and a thermal adhesive resin with one another to obtain a raw material for web. In the step of forming a web, it is preferable that a raw material for web is deposited on a vapor-pervious carrier sheet to form a web.

In the second embodiment, the method preferably comprises a step of adding a deodorizing agent to the web, after completion of a step of forming the web. The step of adding a deodorizing agent to the web may be a step of depositing a mixture of a deodorizing agent and a thermal adhesive resin powder, which have been mixed with each other in air, on the formed web, but is not limited thereto. In this case, the type of the used deodorizing agent and the type of the thermal adhesive resin powder to be mixed can be selected as appropriate, and the mixing ratio of the deodorizing agent and the thermal adhesive resin powder can also be adjusted as appropriate. Moreover, the same type of resin as the thermal adhesive resin used in the step of forming a web may be used herein.

Furthermore, the step of adding a deodorizing agent to the web may also be impregnation of the web with a liquid-state deodorizing agent or application of a deodorizing agent to the web by spraying it. In this case, the timing of adding the deodorizing agent may be after deposition of the web, or may be after the binding step. The timing of adding the deodorizing agent can be selected depending on the purpose or a desired aspect.

In the step of forming the web, a mixture of various types of components such as cellulosic fibers, absorbent fibers and a thermal adhesive resin is deposited on a vapor-pervious carrier sheet placed on a wire fabric (vapor-pervious belt) running along with air serving as a medium. FIG. 5 shows a schematic view explaining a constitution of an apparatus 100 for forming an absorbent sheet with deodorizing functions of the present invention. When the method for producing the absorbent sheet with deodorizing functions is the first embodiment, a fiber mixture obtained by mixing cellulosic fibers, absorbent fibers, a thermal adhesive resin and a deodorizing agent is supplied from a fiber mixture supply unit 130, and it is then deposited on a vapor-pervious carrier sheet on a wire fabric (vapor-pervious belt) 120 to form an air-laid web 13L On the other hand, when the method for producing the absorbent sheet with deodorizing functions is the second embodiment, a fiber mixture obtained by mixing cellulosic fibers, absorbent fibers and a thermal adhesive resin is supplied from a fiber mixture supply unit 130, and it is then deposited on a vapor-pervious carrier sheet 141 on a wire fabric (vapor-pervious belt) 120 to form an air-laid web 131. Then, to the air-laid web 131, a particle mixture obtained by mixing, for example, a deodorizing agent and a thermal adhesive resin powder is supplied from a powder supply unit 133.

Moreover, it may also be possible to mix cellulosic fibers, absorbent fibers, a thermal adhesive resin and a deodorizing agent with one another in air, then to deposit the obtained mixture on a vapor-pervious carrier sheet, then to supply a mixture obtained by mixing a deodorizing agent and a thermal adhesive resin powder in air from a powder supply unit 133 to form a web, and then to form a sheet by a heat treatment unit 135.

The fiber mixture as a web material is three-dimensionally and randomly deposited using an air flow. In that manner, the fibers are deposited to be in a predetermined thickness or more, thereby forming a web (this may also be referred to as a sheet forming step or a web forming step). Such a web forming step is called an air-laid method, and the web formed by the air-laid method is called an air-laid web. As a typical process of an air-laid method, for example, a J & J method, a K-C method, a Honshu method (also called a Kinocloth method or an Oji method) and the like are known.

In the web forming step, for example, a sheet forming apparatus 100 equipped With a conveyor 110, a wire fabric (vapor-pervious belt) 120, a fiber mixture supply unit 130 and a suction box 160 may be used. In such a sheet forming apparatus 100, the fiber mixture supply unit 130 is arranged above the wire fabric (vapor-pervious belt) 120. The wire fabric (vapor-pervious belt) 120 runs, driven by on the conveyor 110 equipped with plural rollers 111. With that, a fiber mixture deposits on the wire fabric (vapor-pervious belt) 120 as assisted by the suction flow from the suction box 160 arranged below the wire fabric (vapor-pervious belt) 120. The fiber mixture drops down along with the air flow and deposits to form an air-laid web.

Above the vapor-pervious belt (wire fabric), a vapor-pervious carrier sheet 141 supplied from a vapor-pervious carrier sheet supply unit 140 is arranged, and the fiber mixture may be deposited on the vapor-pervious carrier sheet 141. Thus arranged, the vapor-pervious carrier sheet 141 enables efficient deposition of the fiber mixture. Examples of the vapor-pervious carrier sheet 141 include a tissue paper and a thermal bond nonwoven fabric. The vapor-pervious carrier sheet 141 may he peeled off after formation of the sheet with deodorizing functions sheet, or may be left as such without being peeled.

In a case where a fiber mixture is deposited on the vapor-pervious carrier sheet 141, occasionally, a thermal adhesive resin may have perviously been sprayed on the vapor-pervious carrier sheet 141. The amount of the thermal adhesive resin to be sprayed is preferably small, and the resin is preferably so sprayed that the amount thereof could be less than the basis weight of the vapor-pervious carrier sheet, and more preferably in an amount of less than 10 g/m². By spraying the thermal adhesive resin in a small amount, the liquid permeability can be kept high and the adhesiveness between the vapor-pervious carrier sheet and the fiber mixture sheet can be enhanced.

In a case where the method for producing the absorbent sheet with deodorizing functions is the second embodiment, a powder supply unit 133 is preferably established further downstream of the fiber mixture supply unit 130. From the powder supply unit 133, for example, a particle mixture obtained by mixing a deodorizing agent and a thermal adhesive resin powder is sprayed. At least a portion of the particle mixture is incorporated into voids in the deposited fiber mixture. Since the fiber mixture is in a state in which it is deposited at a low density due to the suction flow, a part of or the entire particle mixture is incorporated into voids in the fibers of the fiber mixture and is deposited between the fibers of the fiber mixture or the like.

The web formed in the web forming step contains a thermal adhesive resin. Therefore, the formed web is preferably subjected to a heat treatment (binding step). The heat treatment may be carried out, for example, using a heat treatment unit 135 shown in FIG. 5. Through the heat treatment, at least a part of the thermal adhesive resin is melted and is bound to the constituent fibers. The heating temperature applied in the binding step is preferably a temperature at which at least a part of the thermal adhesive resin could be melted. The method of melting at least a part of a thermal adhesive resin to bind fibers is called a thermal bonding method. Specifically, in the present invention, the sheet is formed in combination of an air-laid web method and a thermal bonding method. When an air-laid method is combined with a thermal bonding method in bonding fibers, a sheet having a small density and having many pores can be produced, and the sheet is excellent in absorption performance.

The heat treatment method applied in the binding step includes a hot air treatment and an infrared irradiation treatment. As an example of the hot air treatment, there is mentioned a method of heat treatment where an air-laid web is brought into contact with a through-air drier equipped with a rotary drum having a vapor-pervious periphery (hot air circulation rotary drum system). As another example, there is mentioned a method of heat treatment where an air-laid web is led into a box type drier in which hot air is applied to the air-laid web for heat treatment (hot air circulation conveyor oven system)

After the binding step, a step of compression treatment using a hot roll or the like may be provided for the purpose of controlling the thickness and the density of the sheet. The formed sheet may be stored after wound up in a roll.

In a case where the absorbent sheet with deodorizing functions of the present invention has a surface protective layer and/or a surface contact layer, preferably, the layers are separately formed and laminated in a predetermined order. In lamination, an adhesive resin suitable for adhesion of the layers may be used.

The absorbent sheet with deodorizing functions of the present invention is preferably an absorbent sheet with deodorizing functions produced according to the above-mentioned production method. Such an absorbent sheet with deodorizing functions can exhibit excellent fluid absorbability and deodorizing effect.

### EXAMPLES

The characteristics of the present invention are further specifically described with reference to Examples given hereinunder. In the following Examples, the material used, its amount and ratio, the details of the treatment and the treatment process may be suitably modified or changed not overstepping the spirit and the scope of the invention. Accordingly, the invention should not be restrictively interpreted by the Examples mentioned below.

### Example 1

<Production of Absorbent Sheet with Deodorizing Functions>

The following (a) to (c) were uniformly mixed with an air flow to prepare a fiber mixture.

(a) Cellulosic fibers: pulp fibers (softwood chemical pulp) having a fiber length of 0.05 to 5 mm.

(b) Synthetic fibers: PE/PP core/sheath type composite fibers of 2.2 dtex×5 mm length.

(c) Superabsorbent fibers (SAF): sodium polyacrylate superabsorhent fibers having a fiber length of 6 mm and a fiber diameter of 10 dtex.

Polyethylene (PE) powder was mixed with the following component (d) at a ratio (mass ratio) of 12/10 to obtain a particle mixture of the PE powder and the deodorizing agent.

(d) Deodorizing agent: SHU-CLEANSE 1-411G (manufactured by Rasa Industries, Ltd.).

Subsequently, in the apparatus 100 for forming an absorbent sheet with deodorizing functions shown in FIG. 5, a tissue formed from wood pulp as a raw material (vapor-pervious carrier sheet 141, basis weight: 14 g/m²), which was supplied by a vapor-pervious carrier sheet supply unit 140, was added onto a running wire fabric (vapor-pervious belt) 120 equipped into a conveyor 110.

While aspirating the wire fabric (vapor-pervious belt) 120 by a suction box 160, the PE powder was sprayed at a density of 5 g/m² onto the vapor-pervious carrier sheet 141, and thereafter, from a fiber mixture supply unit 130, the above-described fiber mixture, together with an air flow, was dropped and deposited thereon. At that time, the fiber mixture was supplied, so that the basis weight of the fiber mixture consisting of the above components (a) to (c) could be 244 g/m². Thereafter, the particle mixture of the PE powder and the deodorizing agent was sprayed onto the fiber mixture to result in a density of 22 g/m².

The obtained sheet was passed into a box-type dryer with a hot air circulation conveyor oven system, and was then subjected to a hot air treatment at 150° C. to obtain an air-laid web sheet. After completion of a heating treatment, a breathable film (basis weight: 32 g/m², manufactured by YAMATOGAWA POLYMER CO., LTD.) used as a surface protective layer was laminated thereon with a hot melt resin (5 g/m²). Thereafter, this laminate was treated with a pressure roll to obtain a laminated sheet having a basis weight of 343 g/m² and a thickness of 3.7 mm. While perforating a biaxially oriented PET film with a thickness of 6 μm using hot needles, the PET film was laminated on the laminated sheet according to laminate pressing, so as to obtain an absorbent sheet with deodorizing functions having a surface contact layer as a non-adhering layer, The thickness of the obtained absorbent sheet with deodorizing functions was 2,4 mm.

### Example 2

An absorbent sheet with deodorizing functions was produced in the same manner as in Example 1, except that the deodorizing agent was changed to Mizukanite HP (manufactured by Mizusawa industrial Chemicals, Ltd.).

### Example 3

An absorbent sheet with deodorizing functions was produced in the same manner as in Example 1, except that the deodorizing agent was changed to Taiko CW350BR (manufactured by Futamura Chemical Co., Ltd.) of granular activated carbon.

### Example 4

An absorbent sheet with deodorizing functions was produced in the same manner as in Example 1, except that the deodorizing agent was changed to Taiko SG840A (manufactured by Futamura Chemical Co., Ltd.) of granular activated carbon,

### Example 5

An absorbent sheet with deodorizing functions was produced in the same manner as in Example 1, except that Delnet X550 WHITE-E (manufactured by DelStar Technologies, Inc.) was laminated as a non-adhering layer on the laminated sheet, instead of performing the laminate pressing of a PET film while perforating it with hot needles.

### Example 6

An absorbent sheet with deodorizing functions was produced in the same manner as in Example 1, except that the deodorizing agent was changed to a mixture of Mizukanite HP (manufactured by Mizusawa Industrial Chemicals, Ltd.) and SHU-CLEANSE KD-411G (manufactured by Rasa Industries, Ltd.) (mixing ratio 1/1).

### Example 7

An absorbent sheet with deodorizing functions was produced in the same manner as in Example 1, except that the deodorizing agent was changed to Epoleon X-100 (manufactured by Kyoritsu Seiyaku Corporation), and that the sprayed amount of the particle mixture of the PE powder and the deodorizing agent was changed to 110 g/m².

### Example 8

An absorbent sheet with deodorizing functions was produced in the same manner as in Example 7, except that the deodorizing agent was changed to Epoleon X-110 (manufactured by Kyoritsu Seiyaku Corporation)

### Example 9

An absorbent sheet with deodorizing functions was produced in the same manner as in Example 7, except that the deodorizing agent was changed to Epoleon POW-22 (manufactured by Kyoritsu Seiyaku Corporation).

### Comparative Example 1

An absorbent sheet was produced in the same manner as in Example 1, except that no deodorizing agents were comprised.

### (Evaluation)

### <Absorption Amount Measurement Test>

The sheet produced in Examples and Comparative Examples was immersed in a container containing physiological saline in an amount that the sheet could be sufficiently immersed therein, for 10 minutes. Subsequently, the sheet was left hung for making water drip therefrom for 1 minute, and the sheet weight after immersion was measured. The absorption amount per unit area was calculated by measuring the sheet weight before and after the immersion. The results are shown in Table 1.

### <Deodorizing Test 1>

A deodorizing test was carried out according to the method defined in the certification standards for SEK mark textile products certification standards (Japan Textile Evaluation Technology Council). As odorous components, 5 components, namely, ammonia, acetic acid, acetaldehyde, methyl mercaptan and trimethylamine gas were used, and the sample was tested according to an instrumental analysis (detector tube method) test.

### Instrumental Analysis Test:

An odorous component and a sample of an absorbent sheet with deodorizing functions were put in a container, After 30 minutes, 1 hour, and 2 hours had passed, the remaining concentration of the odorous component was measured. The remaining concentration in the container with an odorous component alone was measured as a blank test concentration, and the decrease rate of the odorous component was calculated according to the following equation, and the tested sample was evaluated according to the following evaluation standards. The results are shown in Table 1.

Odorous Component Decrease Rate (%)=(blank test concentration at each time−sample test concentration at each time)/(blank test concentration at each time)×100
A: The odorous component decrease rate was 75% or more.
B: The odorous component decrease rate was 50% or more and less than 75%.
C: The odorous component decrease rate was 20% or more and less than 50%,
D: The odorous component decrease rate was less than 20%.

### <Deodorizing Test 2>

A dimethyl trisulfide deodorizing test was carried out according to the following procedures.

10 L of air was put into a Tedlar bag, and 10 μL of dimethyl trisulfide was injected therein to prepare a gas having a concentration of about 2 ppm. A sample of an absorbent sheet with deodorizing functions having a size of 10 cm square was put into a separate Tedlar bag and sealed up 10 L of the gas was injected into the Tedlar hag having therein the sample of an absorbent sheet with deodorizing functions, and after a predetermined period of time, the concentration in the bag was measured according to a detector tube method. With that, the odorous component decrease rate was calculated according to the following equation, and the sample was evaluated according to the following evaluation standards. The results are shown in Table 1.

Dimethyl Trisulfide Decrease Rate (%)=(blank test concentration at each time−sample test concentration at each time)/(blank test concentration at each time)×100
A: The dimethyl sulfide decrease rate was 75% or more.
B: The dimethyl sulfide decrease rate was 50% or more and less than 75%.
C: The dimethyl sulfide decrease rate was 20% or more and less than 50%.
D: The dimethyl sulfide decrease rate was less than 20%.

TABLE 1

| | Absorption | | | | | | |
|---|---|---|---|---|---|---|---|
| | Amount | Odorous Component Decrease Rate | | | | | |
| | (g/cm²) | Ammonia | Acetic acid | Acetaldehyde | Methylmercaptan | Trimethylamine | Dimethyl sulfide |
| Example 1 | 7400 | A | A | B | B | A | A |
| Example 2 | 6600 | A | A | | C | A | |
| Example 3 | 6500 | A | A | A | A | A | |
| Example 4 | 6500 | A | A | A | A | A | |
| Example 5 | 7000 | A | A | B | B | A | A |
| Example 6 | 6600 | A | A | B | A | A | A |
| Example 7 | 6500 | A | A | | | A | A |
| Example 8 | 6800 | A | A | | | A | A |
| Example 9 | 6700 | A | A | B | C | A | A |
| Comparative | 6300 | A | A | D | D | B | D |
| Example 1 | | | | | | | |

The sheets obtained in Examples were confirmed to have sufficient absorbability. In addition, the sheets obtained in Examples were also confirmed to have a deodorizing effect for a variety of odorous components. Further, the sheets obtained in Examples were found to exhibit a powerful deodorizing effect for illness-associated odors and a sufficient absorbability fix blood, body fluids, etc.

### REFERENCE SIGNS LIST

1 Absorbent Sheets with Deodorizing Functions
10 Sheet Substrate
13 Resin Layer
14 Surface Protective Layer
18 Surface Contact Layer
S Skin Surface
100 Sheet Forming Apparatus
110 Conveyor
111 Roller
120 Wire Fabric (vapor-pervious belt)
130 Fiber Mixture Supply Unit
131 Air-Laid Web
133 Powder Supply Unit
135 Heat Treatment Unit
140 Vapor-Pervious Carrier Sheet Supply Unit
141 Vapor-Pervious Carrier Sheet
160 Suction Box

1. An absorbent sheet with deodorizing functions, comprising cellulosic fibers, absorbent fibers, a thermal adhesive resin and a deodorizing agent, in a single layer thereof.

2. The absorbent sheet with deodorizing functions according to claim 1, wherein the content of the absorbent fibers is 3% to 50% by mass relative to the total mass of the solid content constituting the absorbent sheet with deodorizing functions.

3. The absorbent sheet with deodorizing functions according to claim 1, wherein the absorbent fibers contain a sodium polyacrylate resin.

4. The absorbent sheet with deodorizing functions according to claim 1, wherein the deodorizing agent contains a component capable of reacting with at least one selected from sulfides, sulfurs and amines.

5. The absorbent sheet with deodorizing functions according to claim 1, wherein the deodorizing agent contains at least one selected from metals, minerals, activated carbons and vegetable extracts.

6. The absorbent sheet with deodorizing functions according to claim 1, wherein the deodorizing agent is a betaine compound.

7. The absorbent sheet with deodorizing functions according to claim 1, further having a resin layer containing a deodorizing agent.

8. The absorbent sheet with deodorizing functions according to claim 1, further having a surface protective layer.

9. The absorbent sheet with deodorizing functions according to claim 1, further having a surface contact layer.

10. The absorbent sheet with deodorizing functions according to claim 9, wherein the surface contact layer is a non-adhering layer.

11. The absorbent sheet with deodorizing functions according to claim 1, which is for covering an area to give off illness-associated odors.
